(19) United States
(12) Patent Application Publication
(10) Pub. No.: US 2004/0186397 A1
(43) Pub. Date: Sep. 23, 2004

(54) Lithotripsy apparatus with an electromagnetic shockwave source triggered by evaluation of an ultrasound B-image

(75) Inventor: Hans-Juergen Haumann, Erlangen (DE)

Correspondence Address: SCHIFF HARDIN, LLP, PATENT DEPARTMENT, 6600 SEARS TOWER, CHICAGO, IL, 60606-6473, US

(73) Assignee: Siemens Aktiengesellschaft

(21) Appl. No.: 10/767,448

(22) Filed: Jan. 29, 2004

(30) Foreign Application Priority Data
Feb. 6, 2003 (DE) 103 04 904.5

(51) Int. Cl. A61B 8/00; A61B 17/22

(52) U.S. Cl. 601/4; 600/439

(57) ABSTRACT

A shockwave lithotripter for destroying a calculus has an ultrasound applicator and an ultrasound device for generating and representing a B-image, an evaluation device for evaluating the B-image and an electromagnetic shockwave generator for generating a shockwave focused at a focus. The evaluation device provides a control signal for the shockwave generator such that shockwave can be triggered only when coincidence between the focus and the calculus is recognized in the evaluation device.

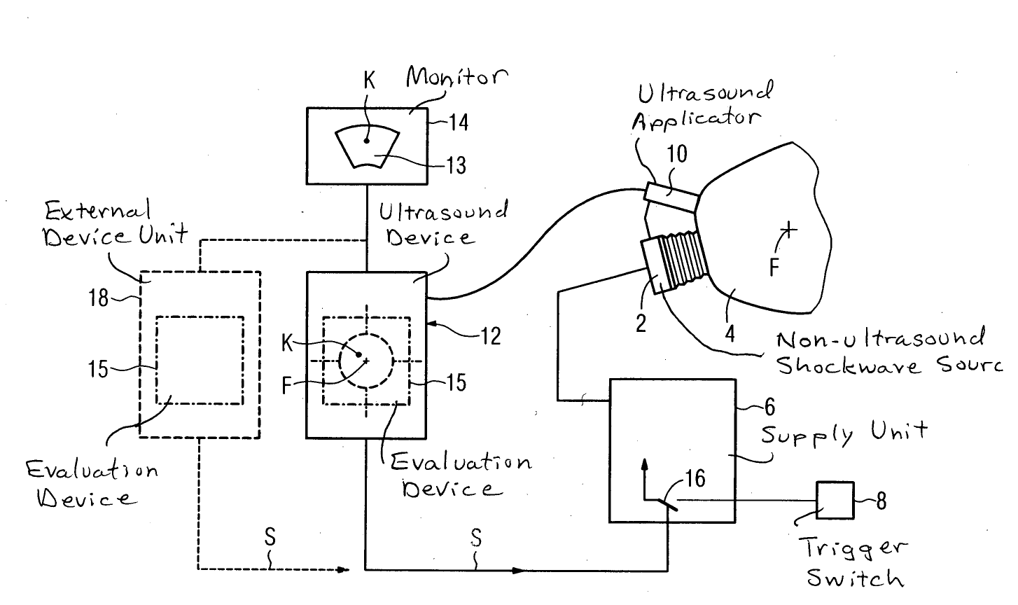

### BACKGROUND OF THE INVENTION

1. Field of the Invention

The present invention relates to a shockwave lithotripter for disintegrating a calculus in the body of a subject of the type making use of ultrasound imaging to control the shockwave triggering.

2. Description of the Prior Art

For disintegrating a calculus situated in the body of a patient, such as a kidney stone, using shockwaves, whether generated ultrasonically or electromagnetically, the shockwave, for the protection of the tissue surrounding the calculus, always must be purposefully focused on the calculus, so that surrounding tissue is not injured and the number of shockwaves that are necessary for the disintegration are kept to a minimum.

U.S. Pat. No. 4,617,931 discloses an ultrasonic shockwave lithotripter which has an ultrasonic converter arranged in the center of the shockwave source, this ultrasonic converter, by means of mechanical pivoting, generating a B-image of the body area surrounding the focus of the shockwave source. This known device makes it possible to precisely position the shockwave source so that it is assured that the calculus acquired by the ultrasonic converter is situated in the focus of the shockwave source. A problem with this known device is that the position of the calculus relative to the shockwave source is not fixed during the therapeutic treatment and can be displaced by up to three cm, even when the patient does not make gross movements, as a result of breathing motions.

In order to assure that a shockwave pulse is triggered only when the calculus is situated in the focus of the shockwave generator, it is known from U.S. Pat. No. 4,803,995 to drive the shockwave generator, prior to each high-energy shockwave pulse, such that it emits an ultrasonic signal of low intensity and receives the appertaining echo signals (A-image). The echo signals are evaluated and a control signal is generated for the shockwave generator dependent on the result of the evaluation. An echo signal of high intensity indicates that a calculus is situated in the focus of the shockwave generator and the shockwave pulse is activated. The shockwave pulse is not activated given the absence of an echo signal. If an echo signal is not acquired three consecutive times, it indicates that the calculus is situated outside of the focus, not as a result of breathing motions, but that a greater deviation is present, due to gross movements of the patient, for example. In this case, the shockwave applicator must be repositioned with using an ultrasound device having an ultrasound converter arranged next to the shockwave source, with a B-image being generated by this ultrasound converter.

In order to shorten the duration of the treatment, U.S. Pat. No. 5,215,091 discloses a further development of the arrangement explained and known from U.S. Pat. No. 4,803,995, such that the measuring signal used for the pulse echo measurement is triggered in shorter time intervals than the shockwave when a prescribed waiting period elapses after the emission of a shockwave. In this known arrangement, the shockwave, therefore, is triggered when the result of the evaluation of the echo signals indicates that the calculus is currently situated in the focus of the shockwave. In contrast to the previously described device, the shockwave pulse not only is suppressed when the calculus is not situated in the focus, but also the shockwave pulse is purposefully triggered when the calculus enters the focus range due to the breathing motions.

A device Is known from European Patent 0 391 378 wherein the ultrasonic converter used for generating a B-Image is also positioned in the center of the shockwave generator, as is the case in the arrangement known from U.S. Pat. No. 4,617,931. It is also assured by this known device that a higher-ranking trigger switch for the lithotripsy mode is disabled during a positioning mode.

It is always required by the devices known from U.S. Pat. No. 4,803,995, U.S. Pat. No. 5,215,091 and European Patent 0 391 378 to operate the shockwave generator in two different modes, i.e., with high power for the stone destruction and with low power for the acquisition of an A-image. The use of piezoelectric shockwave generators is necessary in order to enable such an operating modes. Additionally, the supply device that is necessary for the energy supply of the shockwave source is complex. This supply device must be highly dynamic, or given the use of two different supply units for the two operating modes, these must be connected to the shockwave source in a switchable fashion. Such a changeover is not simple due to the high powers that must be transmitted for generating a shockwave pulse.

### SUMMARY OF THE INVENTION

An object of the present invention is to provide a shockwave lithotripter which assures that a shockwave pulse can be transmitted only when the calculus to be disintegrated is situated in the focus of the shockwave source, and which can be simply realized.

The above object is achieved in accordance with the principles of the present invention in a lithotripsy apparatus having a non-piezoelectric (i.e., non-ultrasound) shockwave source, such as an electromagnetic shockwave source, that is triggerable to emit a shockwave, focused at a focus, for disintegrating a calculus in the body of a subject (in vivo disintegration), an ultrasound applicator, an ultrasound device for generating and displaying a B-image using signals from the ultrasound applicator, and an evaluation device for evaluating the B-image. The evaluation device emits a control signal to the shockwave generator to trigger the emission of a shockwave only when coincidence between the focus and the calculus is recognized in the evaluation device.

Since specialized and highly developed devices are used for locating the calculus, such as an ultrasound device for generating a B-image and an ultrasonic shockwave source for generating a shockwave, the focusing properties of which are optimized for destroying the calculus with point-like accuracy, it is assured that a shockwave pulse can be triggered only when the calculus is situated in the focus of the shockwave source. Furthermore, an electromagnetic shockwave source can be used which generates a shockwave pulse the focusing properties of which are better as a result of the larger focus diameter compared to the focusing properties of a shockwave pulse generated by a piezoelectric shockwave source.

In an embodiment of the invention, the evaluation device is an independent device unit that can be connected to a video output of the ultrasound device. This makes it possible to utilize an ultrasound device that is already present without complex retrofitting being necessary for this purpose.

### DESCRIPTION OF THE DRAWINGS

The FIGURE schematically shows a shockwave lithotripter according to the invention, in an exemplary embodiment.

### DESCRIPTION OF THE PREFERRED EMBODIMENTS

As shown in the FIGURE, the shockwave lithotripter has a non-ultrasound shockwave source 2, such as an electromagnetic shockwave source, which is placed on the body surface of a patient 4 via a water-filled cushion. The shockwave source 2 is connected to a supply unit 6, which is triggered by an external, manually operable trigger switch 8 to supply the shockwave source 2 with the necessary high-voltage pulse. An ultrasound applicator 10, which is also coupled to the main surface of the patient 4, is arranged next to the shockwave source 2 in a fixed spatial position to the shockwave source 2. With the ultrasound applicator 10, a B-image is generated in an imaging ultrasonic device 12 and is displayed at the screen of a monitor 14. In the exemplary embodiment, an evaluation device 15 for evaluating the B-image is integrated in the ultrasonic device 12. The evaluation device 15 evaluates whether a calculus K is situated in the focus F of the shockwave source 2. Dependent on this evaluation, a control signal S is produced which opens a control switch 16, which is connected in series with the trigger switch 8, when the calculus K is situated outside of the focus F of the shockwave source 2 and which closes the control switch 16 when coincidence exists between the focus F and the calculus K. Given a closed control switch 16, a shockwave pulse can be triggered by manually operating the trigger switch 8.

The FIGURE also shows (in broken lines) an alternative embodiment wherein the B-image 13, which is generated by the ultrasound device 12, is digitized and evaluated in an external device unit 18, which is connected to a video output of the ultrasonic device 12 and contains the evaluation device 15.

In this case, a conventional ultrasound device 12 can be used so that an existing lithotripter and an existing ultrasound device for locating the stone can be retrofitted without problems and without exchanging devices.

Although modifications and changes may be suggested by those skilled in the art, it is the intention of the inventor to embody within the patent warranted hereon all changes and modifications as reasonably and properly come within the scope of his contribution to the art.

I claim as my invention:

1. A lithotripsy apparatus comprising:
a triggerable shockwave generator for non-ultrasonically generating and emitting, upon being triggered, a shockwave focused at a focus;
an ultrasound applicator adapted to apply an ultrasound field to a region of an interior of a subject in whom a calculus is present, and to generate electronic signals dependent on interaction of said ultrasound field with the region;
an ultrasound device for generating an ultrasound B-image from said electronic signals; and
an evaluation device for evaluating said ultrasound B-image and for generating a control signal to said shockwave generator to enable triggering of emission of said shockwave by said shockwave generator only if coincidence between said focus and said calculus is recognized in said ultrasound B-image by said evaluation device.

2. A lithotripsy apparatus as claimed in claim 1 wherein said shockwave generator is an electromagnetic shockwave generator.

3. A lithotripsy apparatus as claimed in claim 1 wherein said evaluation device is integrated in said ultrasound device.

4. A lithotripsy apparatus as claimed in claim 1 wherein said ultrasound device has an output at which output at which image signals, representing said ultrasound B-image, are present, and wherein said evaluation device is independent of said ultrasound device and is supplied with said image signals from said output.

5. A lithotripsy apparatus as claimed in claim 1 comprising a manually operable trigger switch for, upon actuation thereof, triggering supply of voltage to said shockwave generator for use in generating said shockwave, and a control switch connected in series between said trigger switch and said shockwave generator and actuated by said control signal from said evaluation device, said control signal closing said control switch when said coincidence exists.
